(19) United States
(12) Patent Application Publication
(10) Pub. No.: US 2015/0202429 A1
(43) Pub. Date: Jul. 23, 2015

(54) Device for Muscle Stimulation

(71) Applicant: Dirk FRITZSCHE, Leipzig (DE)

(72) Inventor: Dirk Fritzsche, Leipzig (DE)

(21) Appl. No.: 14/408,580

(22) PCT Filed: Jun. 28, 2013
(86) PCT No.: PCT/DE2013/100235

§ 371 Date: Dec. 17, 2014

(30) Foreign Application Priority Data
Jun. 28, 2012 (DE) 20 2012 102 393.8
Jan. 21, 2013 (DE) 10 2013 100 586.1

(51) Int. Cl. A61N 1/04; A61N 1/36

(52) U.S. Cl. CPC A61N 1/0484; A61N 1/36003

(57) ABSTRACT

A device for muscle stimulation of the trunk and/or leg musculature by electromyostimulation, comprising a one-part or multi-part suit (2), which is made of an elastic textile fabric and which has an integrated textile-based contact electrode (6, 26) and a textile-based electrical line (7) and bears on a body, wherein the textile fabric is formed as a flat knit (5) in which the textile-based electrical line (7) is formed in a knitted tunnel tube (8), wherein the contact electrode (6, 26) can be electrically connected by means of the line (7), and this contact electrode (6, 26) is designed as a flat knit of conductive thread, which flat knit is designed as intarsia (3) and, on the side facing the body, has an elastic and electrically conductive implantation (4).

### CROSS-REFERENCE TO RELATED APPLICATIONS

This application is the U.S. national stage of International Application No. PCT/DE2013/100235 filed on Jun. 28, 2013, and claims the benefit thereof. The international application claims the benefit under 35 USC 119 of German Application No. DE 102013100586.1 filed on Jan. 21, 2013 and German Application No. DE 202012102393.8 filed on Jun. 28, 2012; all applications are incorporated by reference herein in their entirety.

### BACKGROUND

The invention relates to a device for muscular stimulation of the trunk and/or leg muscle by electromyostimulation.

Electrical muscle stimulation has evolved from a method for specific indications in high-performance sports to standardized techniques in medical rehabilitation towards a rapidly-spreading process in the areas of fitness and “medical fitness”. Effects on the end organ muscles and on the whole organism are described in numerous places and scientifically validated. However, there are hardly any applications that are user-friendly and easy to use, which makes it possible for the target groups to the use the method in home environment and without any assistance.

A known device for muscle training by electrostimulation is known from the publication DE 102 48 235 A1, wherein a variety of body parts, in particular the complete abdominal and back muscles, shoulder, chest, breathing, arm, leg and buttock muscles as well as of the pelvic floor including all the deeper muscle groups are stimulated simultaneously. This is achieved by attaching multiple electrodes to the body, which are arranged on an insulation carrier to be worn preferably over clothing. Such an insulation carrier may preferably comprise of a chest strap and/or a waist strap, which can be connected together for simultaneous use.

The publications U.S. 2002/0133195 A1, AT 401 731 B and DE 202 00 685 U1 show comparable devices.

A disadvantage of these devices for muscle training is that the application is relatively complicated and requires great care to ensure that all electrodes are exactly on the desired areas of the body to rest. Another disadvantage is the complicated wiring of the electrodes. The electrodes can slip, connections can become loose, and there may be areas—due to concave body regions—within which the electrodes do not have complete contact with the body. Finally, a sufficiently effective moisture penetration of underclothing should be noted, whereby training with moist clothing can be felt as uncomfortable. Furthermore, the extensive cabling between the trainee and the stimulation device is partly motion-limiting and uncomfortable.

Other devices for electrical muscle stimulation, for example, according to publication EP 0459945 B1, provide that circular or approximately circular electrodes are placed directly on the skin. This requires precise instructions, such as instructions from a coach or therapist. In particular, plastic adhesive electrodes are used in medical and other fields, wherein these electrodes need to be localized by experienced personnel on the body and at the same time are uncomfortable in contact with skin.

The document US 2007/0049814 A1 discloses a complete system consisting of a suit provided with electrodes, wherein the electrodes are connected to a programmable stimulator device. The connection can be established via cable, but also through wireless. When using a wireless connection, the supply of the electrodes with electric energy appears to be problematic, while a wired device limits the mobility of the user and has risks of accident, especially if connectors are used as designed. In addition, the operation of electromyostimulation through the stimulator device is carried out such that the programs are accessed. These programs restrict the flexibility of the control, so that an individual or customised operation is not possible.

The document WO 2004/006 700 A1 describes a device for electromyostimulation, but which is used mainly for signal extraction from the body (as well as the signal initiation). The electrodes are knitted into a garment, as well as the supply lines (across the carrier material and longitudinally). The electrodes are knitted using conductive, e.g. silver-containing fibres, during a circular knitting process (surface or solid).

Even layered knits (by merging a mirror-image double knit or superposition of two knitting garments) are provided according to the WO 2004/006 700 A1 resulting in more options for placement of electrodes and services.

However, a flat knitting of the textile support or the suit of the electrode is not provided, so that the respective properties are not achievable.

Document U.S. 2007/0049814 A1 discloses a transcutaneous neuromuscular stimulation system, wherein a flexible flat electrode is applied or placed on a suit. A programmable electrical stimulation device is connected to the electrodes. The electrodes comprise a first layer of tissue, a piece of silver-treated material (coated with silver or woven from the silver-coated fibres), which is in contact with the tissue layer, a piece of partially stripped electric wire, which is in contact with the silver. Furthermore, there is a second layer of tissue, which covers the first layer and a contact for electrical connection.

The electrodes may be coated to protect or otherwise prolong its life, whereby there is also an electrically conductive, largely solid or liquid gel in connection with the electrode (either directly on the tissue in contact with the skin, or in a gel reservoir). Each electrode has its head as a lead.

A gel as coating material has a low durability and lifetime, as it can be easily damaged, loosens from the surface or becomes dry. In addition, a solid and reliable connection to the ground is not possible. Furthermore, a flat knitting of the suit and the fabric layer is not provided, so that the respective properties are not achievable.

According to the utility model DE 20 201 1 109 226 U 1, the power supply for an electromyostimulation is indeed integrated in the suit, but the control pulses come from outside, through a control unit connected with the suit. The leads to the electrodes are designed as conductive sewing thread (for example silver-coated PA with insulating sheathing) or sewn into the carrier material or embroidered, but are also knit or woven between layers of the backing layer. The contact elements at the suit are designed as push buttons. The dry electrode is made of silver fabric, which is laminated (plotted, lined) on neoprene backing material (or lycra, spandex). Such superficial lamination can become loose under use, especially when the underlying tissue is stretched. In addition, a flat-knitting of the textile support of the electrode is not provided, so that the respective properties are not achievable.

The publication DE 20 2008 017 860 U1 describes a modified compression stocking modified to perform the electromyostimulation, whereby electrodes are mounted on the compression body. The electrodes are flat or have slight protrusion, have a surface area of 5 to 10 cm² and are made of an electrically highly conductive, resilient material. A control unit is provided on the compression body or a remote control is provided as an alternative. The connection is made through cable or wireless. However, a flat knitting of the textile support of the electrode is not provided, so that the respective properties are not achievable.

An electromyostimulation device with a motion sensor is described in the document DE 10 2006 058 346 A1, wherein the electrostimulation of the movement is dependent and counteracts the wrong movements of the device. The electrode pads are provided with individually controllable electrodes for exact positioning of the lead. A storage device for movement pattern is provided and controller stimulation device, stimulation device and electrode control device are differentiated. A signal connection device between the control device and stimulation device is provided. However, the suit and the fabric backing of the electrodes are not produced by flat knitting, so that the corresponding properties are not achievable.

DE 600 38 341 T2 describes a muscle stimulation as a function of heart rate and other physiological values, for which these values are detected by sensor electrodes and processed in a control device through various complicated algorithms. Both the sensing electrode and the electrode for applying the stimulation pulses, namely the active electrodes, are electrodes of the prior art, including acupuncture electrodes or implanted electrodes. Belts or seats are provided as electrode carriers. A comfortable and ease of use is not available. In addition, a flat-knitting of the suit and the textile substrate of the electrode is not provided, so that the respective properties are not achievable.

However, only a narrowing or widening of the knitted tube would be possible in a circular knit fabric. The partial three-dimensional shape of the knitted product with precise contours and picture adjustment is essential, especially of the inlaid or electrode forms of body and muscle form, which can be realized only with the flat knitting technology in this manner.

Other methods of electromyostimulation, such as Miha-Bodytec.de, Amplitrain cannot be practised without a helping person and therefore not suitable to be used in a domestic environment of a patient. A complex wiring or complicated settings on the control unit for example are reasons against such use.

### SUMMARY

A device for muscle stimulation of the trunk and/or leg musculature by electromyostimulation, comprising a one-part or multi-part suit (2), which is made of an elastic textile fabric and which has an integrated textile-based contact electrode (6, 26) and a textile-based electrical line (7) and bears on a body, wherein the textile fabric is formed as a flat knit (5) in which the textile-based electrical line (7) is formed in a knitted tunnel tube (8), wherein the contact electrode (6, 26) can be electrically connected by means of the line (7), and this contact electrode (6, 26) is designed as a flat knit of conductive thread, which flat knit is designed as intarsia (3) and, on the side facing the body, has an elastic and electrically conductive implantation (4).

### DETAILED DESCRIPTION

The invention has the objective of providing a device that will help in harnessing the undisputed benefits of the method of electrostimulation to patients in their home environment. It is further provided to enhance the acceptance and to avoid application problems in electromyostimulation. A suitable device for muscle stimulation of trunk and leg muscles should be offered, which can be used flexibly and comfortably and is safe and easy to use.

The objective of the invention is achieved by a device for muscular stimulation of trunk and/or leg muscles by electromyostimulation, comprising at least one integrated textile-based contact electrode and at least one textile-based electric wire adjacent to a body, one or more parts of a suit made of elastic fabric. The fabric is, preferably designed as a flat knitted fabric; preferably as a single-surface flat sew knitted without seams and without the need for parts. A three-dimensional adjustment of the knit, for example by using different knit based on inlay surfaces, to the contours of the body is possible due to the flat knit such that the suit is close to the body completely. The three-dimensional flat knit, contrary to circular knit, facilitates a partial compression and an optimal adjustment to the body form.

Moreover, electrical wiring, or at least one lead is integrated in the fabric surface, by means of which the contact electrodes can be connected electrically. These services are preferably based on fabrics, i.e. produced by textile technology method using electrically conductive threads, preferably knitted. Knitting can be done separately, with the aim of placing the lines into the tunnel pipes. However, it is intended to knit the flat knitted fabric of the suit, the tubes and the lines in a single operation.

However, other types of lines such as metal wires or strands are also intended. These lines are arranged in a preferred embodiment in a fully closed circular channel knitted as tunnel pipes, or knitted separately or processed by other methods. The tunnel is preferably designed as a flat knit tunnel. The lines are thus directly on the fabric. The surfaces of the inserted electrical lines are then connected in a manner such that an electrical contact is done beyond the individually knitted surfaces. Thus, the contact electrodes can be electrically contacted and an electrical connection between a contact site on the suit, where the stimulation signal is supplied from the stimulation unit and the contact electrode is possible. Contacting the electrical line with each other is specifically required, when the suit consists of a multiple flat knitted fabric surfaces that must be joined together, e.g. by sewing. The electrical lines are contacted with the electrically conductive connection when the suit is assembled, e.g. sewn, pasted or clamped, wherein the respective electrical connection remains active. Correspondingly, the connection is established between the line and contact electrode.

Regardless, it is provided according to the invention to use some other means of power and signal transmission, for example through the outside of the suit or in the textile composite, such as a tissue, guided conventional cables or wires.

Furthermore, one or more contact electrodes are designed at least as a single-layer flat knit built as inlay, which consists of partially conductive material by using conductive threads, and with a stimulation layers on the body side that is electrically conductive. The surface of the flat knit forming the suit is described as flat knit, wherein at least partially conductive threads such electrically conductive yarn is used for manufacturing such knit. The conductivity may be based on chemical effects (such as carbon polymers) as well as on physical effects (such as metals, metal alloys) and combinations thereof (for example, metallic coatings synthetic) based. The conductive yarn is also used in the manufacture of the electric wire. The inlay can be worked onto one or both surfaces of the surrounding flat-knitted fabric, or alternatively of another knitted fabric or knitted fabric and penetrate into it. Alternatively, the inlay can be present in the full cross section of the surrounding flat-knitted fabric and these penetrate completely.

In particular, the contact electrode is provided in a preferred embodiment of the invention at least at the body-facing side with a flexible, electrically conductive implant as the stimulation of the skin-contacting layer. This means that not only a coating is present, but the implant material extends both on the surface of the inlay of the contact electrode, and at least partially into the interior, for example, migrates between the meshes of a flat-knitted fabric. This will be extremely durable and achieve a secure electrical contact. Extension of the flat-knitted fabric is transferred gently to the implant so that a release from the fabric is avoided permanently and thus assures a high wearing comfort. The implantation covers the inlay at least partially, but can also extend over the inlay, except beyond the flat-knitted fabric.

A polymer is preferably used for implantation, wherein the implantation takes place for example by means of pressing power, heat or other similar appropriate procedure. By using a polymer—preferably rubber—a passive hydrogenation by local moisture developing on the skin, thus body moisture is created. The electrodes therefore require no additional excitation by moisture cushion and no increase in the electrical conductivity by means of a gel or gel reservoirs.

The soft cushion-forming coating is soft to the skin and distributes the current evenly to the skin due to their electrical properties. Thus, there are no unpleasant local current peaks and the power entering the skin remains comfortable for the user. The electrically conductive coating only represents an additional element of the electrode that is complete and functional from the textile process and has suitable electrical properties. In particular, favourable wearing properties and the aforementioned advantages are achieved, when the coating is made electrically conductive dispersion or an electrically conductive polymer, because these materials abut softly against the skin.

Electrically conductive polymer compositions are considered as material for the contact electrodes and/or stimulation layer, whereby such compositions are arranged on the textile structures and penetrate into the latter. Preferably an electrically conducting rubber is provided, which forms the textile based electrode on the fabric surface, which is preferably designed here as a flat knitted fabric. However, there are also other kinds of fabric, such as knit or circular knit fabrics, as well as other types of electrical conduction, such as insulated strands, which are taken into consideration.

Further advantages of the device according to the invention are due to the fact that only a single fabric surface is present in the flat knit, but these can be designed in different knit forms depending on the required three-dimensional contours. Here, a formation according to the forms of the body and muscle is made possible, whereby the electrode contours accurately reflect the muscle and body geometry in the situation and therefore an optimal match and conditioning the electrode to the skin can be realized. Further, the electrical line is surrounded on the electrical connection of the contact electrode by means of a knitted-in tunnels and thus it is insulated, protected and is not visible. The tunnel or the tunnel pipes is preferably formed together with the flat knitted fabric, of the tunnel tube and the electric wire to each other, for example, knitted in a single operation. Alternatively, it is also provided to subsequently introduce the electric line into the tunnel, especially if it is not a knitted line, but a strand or similar.

In addition, the contact electrode has an implanted polymer, which causes a passive hydrogenation toward the skin surface in the direction of the knitted fabric surface, and an optimal electrical conductivity secured by a low contact resistance of the electrode surface to the body and at the same time increasing the comfort of the garment. In addition, the specific production of all textile elements in the process of flat knitting leads to a high efficiency in the production of the inventive device for muscle stimulation by electromyostimulation.

Any garment having the features and functions of the invention should be considered as a suit. It may be possible that the complete suit is used to stimulate the trunk and leg muscles, or individual parts such as a top or a pair of tights to stimulate specific areas of the body such as the torso or legs, individually.

The contact electrodes are positioned with the aid of the suit at designated points on the body to initiate stimulating electrical impulses there. The contact electrodes are connected with the stimulation unit in a manner that enables electrical conduction.

However, the operation is not done directly via the stimulation unit, wherein such a solution is also covered by the invention, but preferably via a control unit in a comfortable manner. The signals generated by the control unit for entering the operating commands are transmitted to the stimulation unit via the communication unit. Thereby, the inventive device allows both simple and flexible operation by the user, which can comprehensively influence the type and strength of the stimulation of his muscles, use predefined programs or can adjust the stimulation individually. This fulfils the desire to achieve positive, health-promoting effects, such as with a regular conventional training, but with less investment of time and quick implementation.

A suit that is close to the body and is made of one or more parts of an elastic textile fabric with integrated textile-based electrode contact and electrical lines integrated in the fabric surface is of a major advantage. A body-fitting, elastic suit increases the well-being and keeps the user warm as optimal conditions for a successful training. Based on the preferences of the user or circumstances, under which the device is used, the suit can be of single piece.

It is a major advantage, if the electrical lines are fabric based and thus are integrated functionally into the fabric without affecting the elasticity and other characteristics of the suit. This can be done for example by metal-coated, preferably insulated synthetic fibres, which are processed together with the other fibres forming the fabric. Thus, the signals from the stimulation unit can be passed to the contact electrodes through the textile fabric, which forms the suit.

It is also very comfortable, if the tights with at least one circumferential contract electrode is arranged in the area of upper and lower leg, however a contact electrode in upper and lower leg is preferred. In this way the leg muscles can be trained specifically and extensively without having to wear a full suit.

Alternatively the pressure-elastic fabric spacer structures and/or elastic, vertical filament stocking are designed as a moisture storage core. This ensures a particularly good electrical conductivity. Alternative embodiments are characterized as previously described by a contact electrode, which is due to the material composition that does not depend on a humidification.

However, all the contract electrodes, at least a part thereof, are supplied by a stimulation unit with stimulation current as controllable power supply. The multi-part, preferably two-piece version of the suit according to the invention is designed such that each part has its own stimulation unit. Alternatively, parts of the suit have electrical connections to the part that covers the stimulation unit. A controller is used to adjust the functions of the stimulation unit.

Particular advantages may be observed if the stimulation unit is connected with the suit such that it can be easily detached. In this way the suit can be worn without any impairment when no stimulation training is being performed. In addition, a stimulation unit can be used by many users each one having a customised suit.

This has proven to be advantageous when at least a metal or metallized push button is provided for connecting the stimulation unit with the suit. Such a connection represents a durable and safe electrical connection. Multiple contacts are provided as a preference. The stimulation unit is electrically contacted through push buttons and is uniformly attached to the suit.

Alternatively or in addition to the use of push buttons, a metallized Velcro fastening element is provided. A hook and loop fastener is simple and flexible to use in such a way that it is attachable to different places. If the stimulation unit, for example, has the hook side, it can be fitted on a larger area of the suit with fleece. For example, this can be designed such that the contacts extend as an open conductor tracks vertically to suit and hence the stimulation unit can be attached vertically to increase the convenience of the user in different positions.

As another alternative, at least a magnetic contact for connection is provided. Magnetic contacts provide a reliable electrical connection and can be detached quickly and easily when required, for example, when the user encounters an obstacle when moving with the suit. Thus, accidents and damages to the suit can be prevented.

Preferably, the stimulation unit has an accumulator for power supply. The accumulator provides the energy, the electrical current for operating the contact electrodes, which then pass the current to the body. Thus, energy can be quickly recharged and the stimulation unit is ready for use again when an accumulator, which is fixed or connected interchangeably with the stimulation unit, is used. According to the invention it is also planned to use rechargeable batteries or other power sources such as a generator, this preferably in conjunction with an at least temporarily active memory.

Particular advantages are derived when the control unit comprising of a touch-sensitive screen to control the activity of the contact electrodes is used as a graphical user interface, and the operating unit is spaced out from the suit. A spaced control unit ensures that the suit and elements on the suit have a minimum weight while ensuring optimal operation. This is especially useful for elderly or people with reduced mobility, whereby they do not have to change the stimulation unit by themselves.

In a preferred embodiment, retrievable programs to control the activity of the contact electrode for the supply of electrical pulses are stored in the control unit. Thus, the stimulation unit is smaller and is of lighter built and the information capacity of the system electromyostimulation can be transferred wholly or for the most part to the operating unit. Preferably, this is arranged away from the suit and the stimulation unit and is not being worn by the user while he completes the stimulation training.

The high efficiency with respect to the executable programs provided by the control unit permits processing of automatically generated input data in an alternative embodiment form. In the case of a bidirectional communication between stimulation and control unit, values such as body temperature, skin resistance or physiological currents can be determined in real time with sensors in the suit in order to arrive at a more refined and individualized training process.

Alternatively or additionally, the activity of at least one individual contact electrode is separately controllable. Regardless of the diverse programmatic options offered with the operating unit, it should also be possible for an individual to manually navigate to the contact electrodes. This allows the user to quickly respond to individual needs or wants, without calling an appropriate program or even creating one.

Special benefits can be seen when the communication unit is configured for wireless signal transmission and a fixed cable connection between the stimulation unit and control unit can be omitted. This increases the freedom of movement of the user and accidents such as by tripping or snagging on the cable are prevented. In addition, the user can easily pursue leisure activities or other activities while he completes the training.

It has proven to be very advantageous, when the communication unit is designed for transmitting signals according to Bluetooth standard. As it is an established communication technology, the corresponding components are functionally reliable, readily available and inexpensive. In addition, this is a transmission standard which requires only very low levels of output, so that the undesirable emission of electromagnetic radiation is minimal despite the proximal position. The Bluetooth transmission according to class 3 with 10 m range has a maximum transmit power of 1 mW compared to 2000 mW in mobile phones.

Advantages result from a communication unit, which is designed for mono-directional signal transmission of the control signals from the control unit to the stimulation unit. Bidirectional transmission, as described above, offers extended options to influence the stimulation algorithm taking into account physiological reactions. However, a mono-directional (or asymmetric with Bluetooth) communication unit is simple, inexpensive and has a long battery life, because the part in the stimulation unit is designed only as a receiver.

A particularly advantageous and cost-effective combination of the control unit and the communication unit is achieved by using commercially available tablet PC or a smart phone, which is controlled by a program planned for device according to the invention. Thus, there is a multifunctional operating unit that has a touch sensitive screen and at least one device for wireless communication.

### BRIEF DESCRIPTION OF THE DRAWINGS

Further details, features and advantages of the invention will become apparent from the following description of an embodiment with reference to the accompanying drawings. The drawings show the following:

FIG. 1 schematically shows a schematic diagram of one embodiment of a suit of a device for complex electromyostimulation;

FIG. 2 schematically illustrates a schematic diagram of an embodiment of a rubber electrode of a device for complex electromyostimulation;

FIG. 3 schematically shows a schematic diagram of one embodiment of an apparatus for complex electromyostimulation;

FIG. 4 schematically shows a schematic diagram of an embodiment of a stimulation unit and

FIG. 5 schematically shows a schematic view of another embodiment of an apparatus for complex electromyostimulation.

### DETAILED DESCRIPTION OF THE PREFERRED EMBODIMENTS

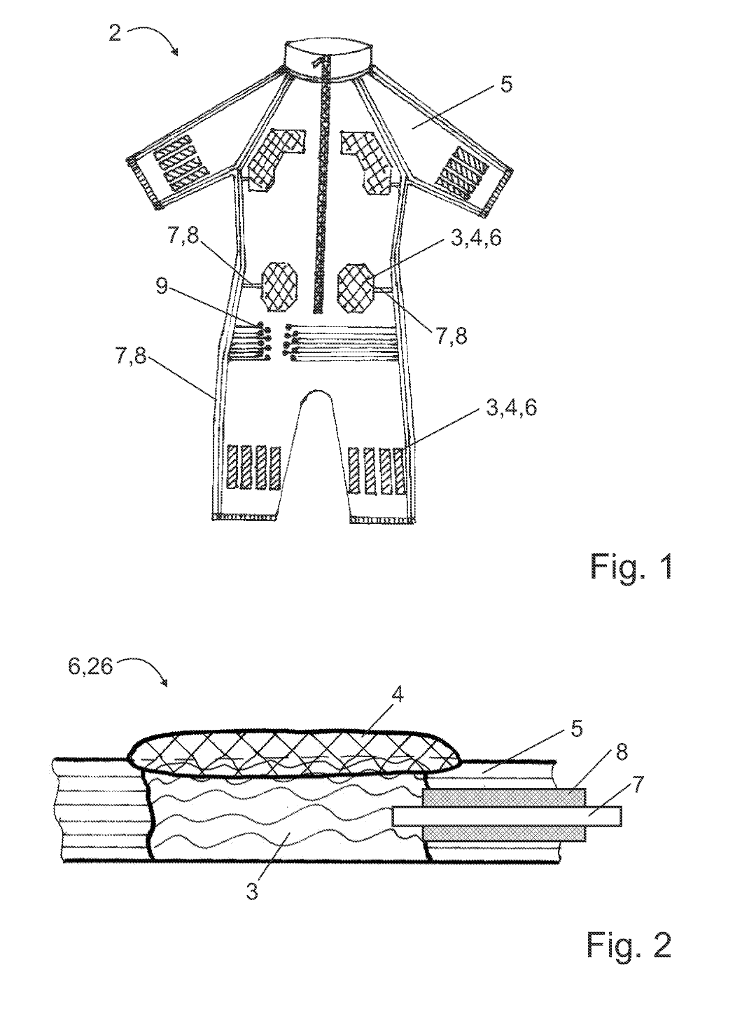

FIG. 1 shows schematically an illustration of one embodiment of a suit 2 for use in a device according to the first complex electromyostimulation. The suit 2 is flat knitted and has contact electrodes 6 provided on different points on the front and the back side. These consist essentially of a flat knitted inlay 3 by using conductive thread, incorporated into the flat-knitted fabric 5 of the suit, and have an implantation 4 towards user's skin. Implantation 4, preferably consisting of rubber, is connected in such a way with the inlay 3 that a mechanically strong and electrically safe conductive connection is ensured. The manufacturing of the suit 2 in flat knitting process allows an optimum adaptation to the body shape.

The electrical connections of the contact electrodes 6 is done by means of electric leads 7, which are also flat knitted in the preferred embodiment of electrically conductive threads and are also knitted into tunnel 8. This tunnel tube 8 protects the electrical cable 7 against mechanical damage, is used as insulation and at the same time serves as a favourable visual impression of the suit 2, for example when used as a decorative element.

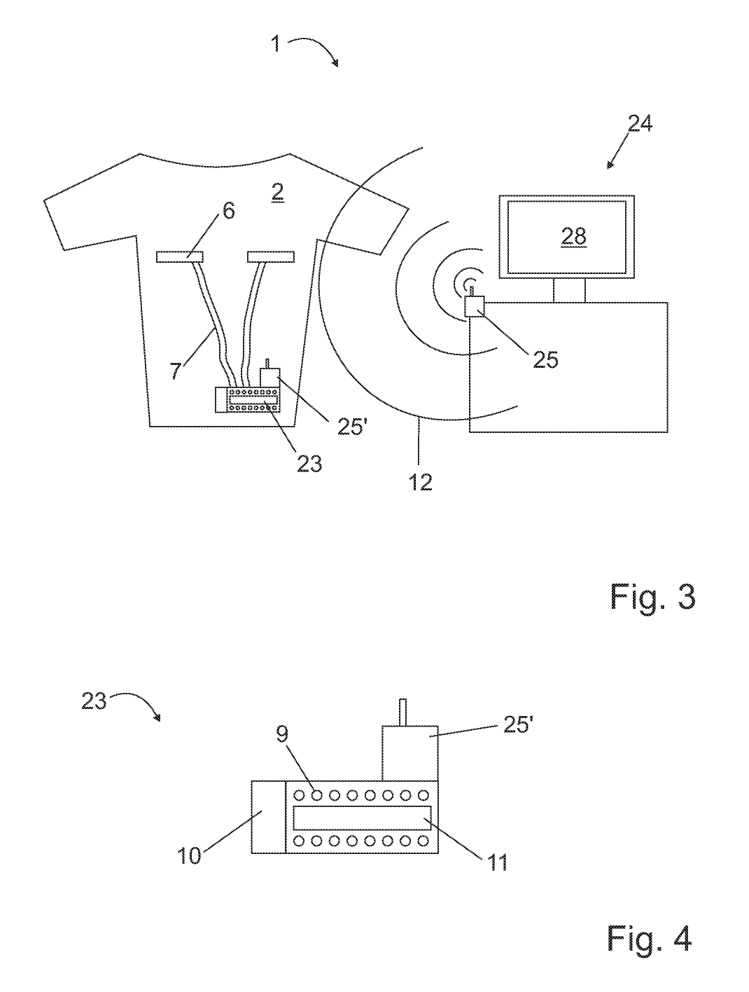

The electrical lines 7 are connected to a metallized push button 9 which is attached to the outside of the suit 2 and used to connect to the stimulation unit 23 (see FIG. 3, 4 or 5).

FIG. 2 shows schematically a sectional view of an embodiment of a contact electrode 6, 26 including the electrical connection, which is introduced into the flat knitted fabric 5 of a suit according to the invention. A flat knit inlay 3, which is manufactured using electrically conductive threads, is incorporated into the flat-knitted fabric 5 forming the suit. In the present embodiment, the inlay penetrates the flat knit 5 completely and a partial penetration is also provided as an alternative. Furthermore, also preferably knitted, but also metallic conductors or any other appropriate means of electric line 7 is supplied in the flat-knitted fabric 5, which in turn runs into a protective and insulating tunnel tube 8. The tunnel tube 8 is preferably made in the flat knitting process. This allows the suit or a suit part to be manufactured in a single operation, as far as the technical textile design is concerned.

On the inlay 3 there is an electrically conductive material, preferably rubber, that is applied in such a way that there is an intense connection between the inlay 3 and 4 of the implantation occurs in the inlay 3 and implantation 4, whereby inlay 3 and implant 4 penetrate partially. This ensures high mechanical strength against peeling, a very good electrical connection between inlay 3 and implant 4 as well as matching extension characteristics between flat-knitted fabrics 5, inlay 3 and 4 implantation.

The surface of the implant 4 is intended for installation on the skin, thereby ensuring a large-scale transmission of electrical stimuli to the skin and also causes mild local moisture, which improves the transfer of current from the contact electrode 6, 26 to the skin.

FIG. 3 schematically shows a schematic view of an embodiment of an inventive device 1 for complex electromyostimulation with the suit 2, where the stimulation device 23 is arranged. Another main component is a control unit 24 Bluetooth transmitter 25 as part of the communication unit. This communicates with the Bluetooth transceiver 25, connected to the stimulation unit 23.

The stimulation unit 23 is connected via electrical leads 7 to the contact electrodes 6 that are arranged at or in the fabric of the suit 2. Accordingly the modulation pulses, electric currents are transmitted to the contact electrodes 6 and there transferred into the body of the user due to the contact with the skin. For this purpose, the contact electrodes 6 are formed so that a low contact resistance is ensured to the skin on the entire surface of the contact electrodes 6. Thus, the intended dose stimulation is transmitted, but without irritating the nerves of the skin and causing unpleasant stinging. This is accomplished in the preferred embodiment by the previously described implant with an electrically conductive rubber. The electrical connection is preferred over the textile-based lines.

The control unit 24 has the touch screen 28. On this one, the user-optimized user interface is displayed. Likewise, a contextual change is possible. The control unit 24 arranged away from suit 2 and stimulation unit 23 permits more options when compared to a portable control unit, not the least program options, for example it provides computer integrated in the control unit with regard to computing power or connection to a network.

The wireless communication between the Bluetooth transmitter 25 and the Bluetooth receiver 25′ is represented by the stylized Bluetooth signal 12.

FIG. 4 schematically shows a schematic diagram of an embodiment of a stimulation unit for the mechanical and electrical connection at the same time with a suit or a pair of tights shown in FIG. 3, exemplified in the preferred embodiment with metallized push button 9, a total of 16 pieces. The counterparts of the push buttons 9 are located on the suit. To connect the contacts of the suit with the elaborated as pushbuttons 9 mating contacts in the stimulation unit 23, the push buttons are pressed into each other 9. The stimulation unit 23 further comprises the battery 10 which provides power and is preferably carried out to the external charging removable. Furthermore, the stimulating unit 23, a control board 1 to 1, which converts the 25′ received from the Bluetooth receiver signals in a manner such that stimulation currents, electrical pulses flow through the push-buttons 9. Alternatively, simple programmatic steps in the control board 1 1 were carried out to relieve the communication unit.

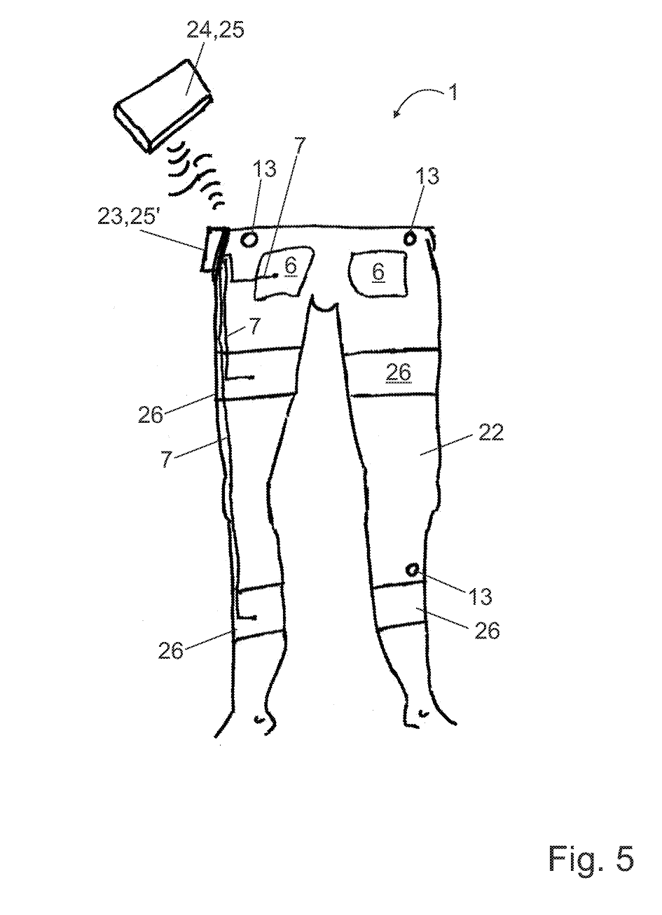

FIG. 5 schematically shows a schematic view of another embodiment of a device for complex electromyostimulation as electrical tights 22. It is a stocking 22, to which electrically conductive surfaces such as inlays or flat knits are integrated as contact electrodes 6, 26, wherein circumferential contact electrodes 6 are cover upper and lower leg musculature. The tights can optionally cover the entire leg and foot or enclose only to the extent that even the circumferential contact electrodes 26 are fixed to the lower legs in their position through tights 22.

The electrical connection of these contact electrodes 6, 26 to the stimulation unit 23 is made by electric lines 7 integrated in the fabric. The stimulation unit is preferably inserted in the hip into the fabric or otherwise attached there permanently or temporarily. There are also sensors 13 integrated in the fabric, preferably as a signal transducer for recording the ECG. Preferred locations for the installation of sensors 13 are the right hip, left hip and/or lower leg. The stimulation currents can be influenced by processing the ECG in a processing unit integrated in the stimulation unit 23 or control unit 24. Thus, a stimulation mode that is synchronous with the heart and not synchronous with the heart can be selected as options. The communication between control unit 24 and stimulation unit 23 is done preferably over wireless via communication unit 25, 25′.

The fabric-based, electrically conductive surfaces of the contact electrodes 6, 26 trained as stimulation layer are galvanised with an implant made of rubber, preferably made of a ionised polymer. The stimulation layer is in a position to transfer the current to human skin without additional moisture.

The stimulation unit 23 is controlled by a control unit 24. Preferably, this is a tablet PC or a smartphone, which is driven by special software. Based on the wireless signal transmission through integrated communication unit 25, 25′ that is present as default in such devices, all cables that inhibit easy and safe operation can be dispensed with. The wearing comfort thus allows its use in daily life and an assisting person is not necessary.

Other advantages of the device according to the invention are in designing acknowledged and validated therapy options such that they are user friendly and are accepted by the target group and can be used without any external help. Thus, there is a lower re-admission rate of the target group, especially heart failure patients, and there is positive effect on the underlying disease.

### LIST OF REFERENCE NUMERALS

1 Device for complex electromyostimulation
2 Suit
3 Inlay
4 Implantation
5 Flat knit
6 Contact electrode
7 Electrical line
8 Tunnel pipe
9 Metallised push button
10 Accumulator
11 Control board
12 Bluetooth-Signal
13 Sensor
23 Stimulation unit
22 Tights
24 Control unit
25 Communication unit, Bluetooth-Transmitter
25 Communication unit, Bluetooth-Receiver
26 Circumferential contact electrode
28 Touch-sensitive screen

1. Apparatus for muscular stimulation of body and/or leg muscles by electrostimulation, comprising of at least an integrated textile-based contact electrode (6, 26) and at least one textile-based electrical line (7), body covering suit that has one or more parts and is made of an elastic fabric (2), characterized in that, the fabric is designed as a flat-knitted fabric (5) in which at least one electrical conduit (7) is integrated or is placed in a knitted tube (8), wherein the contact electrode (6, 26) can be electrically connected using the wire (7) and is designed as a single-layer inlay (3) of flat-knitted fabric that has partially at least electrically conductive yarn, that has an elastic, electricity conducting implant (4) an electrically conductive rubber insulated wire placed on the flat-knitted fabric (5) on the side that is in contact with the body.

2. A device according to claim 1, wherein said flat-knitted fabric (5) of the suit (2) is designed as a single layer.

3. A device according to claim 1, wherein the electrical line (7) is at least partly made of electrically conductive yarn that has been manufactured according to a textile industrial process.

4. Device according to claim 1, wherein the electrical line (7) is knitted.

5. Device according to claim 1, wherein said flat-knitted fabric (5), the tube (8) and the electrical line (7) are integrated with each other.

6. Device according to claim 1, wherein the electrically conductive yarn of the inlay (3) or line (7) is made of a carbon polymer, or a metal or a metal alloy, or comprises a carbon or metal coating.

7. Device according to claim 1, wherein the garment (2) of multiple flat-knitted fabrics (5), whose electric lines (7) are connected to the electrically conductive source during assembly of the suit (2).

8. Device according to claim 1, wherein the single-layered, flat-knitted fabric designed as an inlay (3) covers the entire cross-section of the flat knitted fabric (5) or is included on one or both sides of the surface of the flat-knitted fabric (5).

9. Device according to claim 1, wherein the electrically conductive rubber wire is provided as the electrically conductive implant (4), which is embedded in the surface of the flat-knitted fabric designed as an inlay (3) that the flat-knitted fabric is at least partially covered.

10. Device according to claim 1, wherein the suit (2) is at least partially designed as tights (22) and has at least one circumferential contact electrode (26) in the region of the upper or lower leg.

11. Device according to claim 1, wherein the stimulation unit (23), a control unit (24) and an operating unit (24) and the stimulation unit (23) for signal transmission connecting the communication unit (25, 25′) are provided.

12. A device according to claim 11, wherein the control unit (24) as graphical user interface comprising a touch sensitive screen (28), and for controlling the type of the contact electrodes (6, 26) is provided, and wherein the control unit (24) of the suit (2) is spaced apart.

13. Device according to claim 11, wherein the programs for controlling the activity of at least one contact electrode (6, 26) are stored in control unit (24) and the stimulation unit (23) for the controllable power supply of the contact electrodes (6, 26) via the electric lines (7) is between the stimulation unit and the contact electrodes on the basis of programs for controlling the activity.

14. Device according to claim 11, wherein the stimulation unit (23) is connected to the suit (2) in a detachable way.

15. Device according to claim 11, wherein the communication unit (25, 25′) is designed for wireless signal transmission.
